(19) United States
(12) Patent Application Publication
(10) Pub. No.: US 2016/0222095 A1
(43) Pub. Date: Aug. 4, 2016

(54) METHOD OF ADMINISTRATING IgC COMPOSITIONS TO PROVIDE SUSTAINED LEVELS OF AMINO ACIDS

(71) Applicant: The Lauridsen Group, Inc., Ankeny, IA (US)

(72) Inventors: Eric Weaver, Ankeny, IA (US); Abigail Henderson, Ankeny, IA (US); Christopher Detzel, Ankeny, IA (US)

(73) Assignee: The Lauridsen Group, Inc., Ankeny, IA (US)

(21) Appl. No.: 15/011,175

(22) Filed: Jan. 29, 2016

Related U.S. Application Data
(60) Provisional application No. 62/109,417, filed on Jan. 29, 2015.

(51) Int. Cl. C07K 16/06

(52) U.S. Cl. CPC C07K 16/06

(57) ABSTRACT

The present invention provides methods for providing peak plasma amino acid concentrations through administration of an oral dose of at least 20 g of immunoglobulin concentrate.

### CROSS REFERENCE TO RELATED APPLICATION

This application claims priority under 35 U.S.C. §119 to U.S. Patent Application No. 62/109,417, filed Jan. 29, 2015, the content of which is incorporated hereinto by reference.

### BACKGROUND OF THE INVENTION

Proteins are essential nutrients needed to maintain the overall health of our bodies. Amino acids are the building blocks of protein and, in combination with nitrogen, form several different proteins. There are two basic types of amino acids: essential and nonessential. The former is not produced within the body so they must be obtained from outside sources. The latter is produced internally within the body. Both types perform specific functions in the body.

Leucine, glutamate, tryptophan, and arginine are among the important “bioactive amino acids”, and participate in many important and diverse biochemical reactions associated with the normal physiology of the organism.

Leucine is an essential amino acid and has a chemical composition identical to that of isoleucine, but its atoms are arranged differently resulting in different properties. Leucine is obtained by the hydrolysis of protein by pancreatic enzymes during digestion and is necessary for optimal growth in infants and children and for the maintenance of nitrogen balance in adults. Leucine lowers elevated blood sugar levels and is necessary in promoting the healing of bones, skin, and muscle tissue. Supplements and protein powders that contain leucine are used extensively by bodybuilders and other athletes to promote muscle recovery. It also works to increase endurance and enhance energy.

Tryptophan is also an essential amino acid which is naturally found in animal and plant proteins. Tryptophan is important for the development and functioning of many organs in the body. After absorbing L-tryptophan from food, our bodies convert it to 5-HTP (5-hydroxytryptophan), and then to serotonin. Serotonin is a hormone that transmits signals between nerve cells and causes blood vessels to narrow. Changes in the level of serotonin in the brain can alter mood.

Due to the beneficial effects of amino acids, and the essential amino acids tryptophan and leucine in particular, it would be desirable to find means for increasing and/or sustaining amino acid levels in the body.

It is therefore a primary objective of the present invention to provide an improved method and means of improving amino acid levels in the body.

It is another objective of the present invention to provide a method and means of sustaining levels of amino acids in the body.

These and other objects of the invention will become apparent from the detailed description of the invention which follows.

### SUMMARY OF THE INVENTION

The present invention relates to the administration to animals of at least 20 g of an IgC composition to animals to provide a dose of at least 10 g of IgG composition. The present inventors have surprisingly determined that once orally administered, the amino acids maintain their peak concentrations for a time period of at least 15 minutes post administration and up to about 180 minutes.

### BRIEF DESCRIPTION OF THE DRAWINGS

FIGS. 1-24 provide charts showing experimental results illustrating the mean amino acid concentration in plasma (nmol/mL) over a time course following administration of placebo or serum bovine immunoglobulin (SBI) at 5 g, 10 g, or 20 g per day. The amino acid concentrations shown in each figure are as follows:

FIG. 1 provides the mean total amino acid concentration.

FIG. 2 provides the mean essential amino acid concentration.

FIG. 3 provides the mean alanine concentration.

FIG. 4 provides the mean arginine concentration.

FIG. 5 provides the mean asparagine concentration.

FIG. 6 provides the mean aspartic acid concentration.

FIG. 7 provides the mean citrulline concentration.

FIG. 8 provides the mean cystine concentration.

FIG. 9 provides the mean glutamic acid concentration.

FIG. 10 provides the mean glutamine concentration.

FIG. 11 provides the mean glycine concentration.

FIG. 12 provides the mean histidine concentration.

FIG. 13 provides the mean isoleucine concentration.

FIG. 14 provides the mean leucine concentration.

FIG. 15 provides the mean lysine concentration.

FIG. 16 provides the mean methionine concentration.

FIG. 17 provides the mean ornithine concentration.

FIG. 18 provides the mean phenylalanine concentration.

FIG. 19 provides the mean proline concentration.

FIG. 20 provides the mean serine concentration.

FIG. 21 provides the mean threonine concentration.

FIG. 22 provides the mean tryptophan concentration.

FIG. 23 provides the mean tyrosine concentration.

FIG. 24 provides the mean valine concentration.

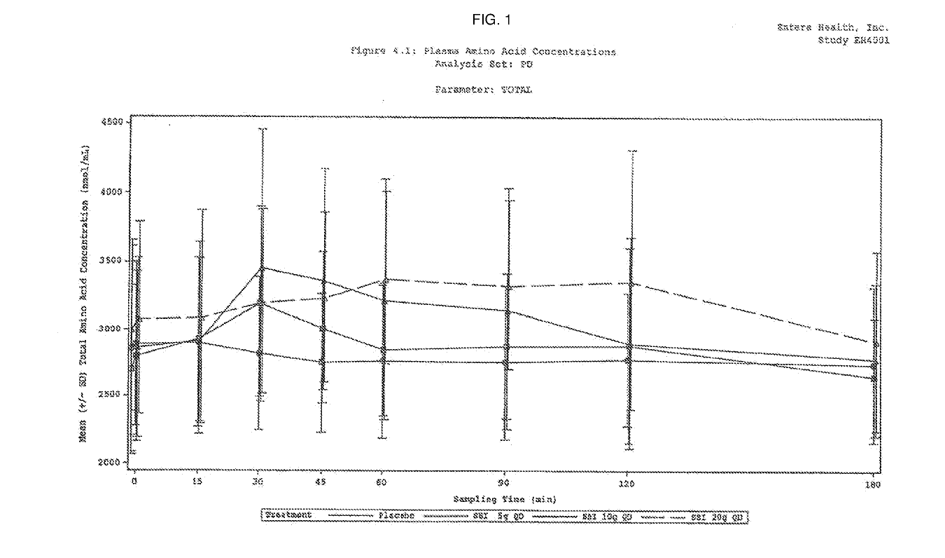

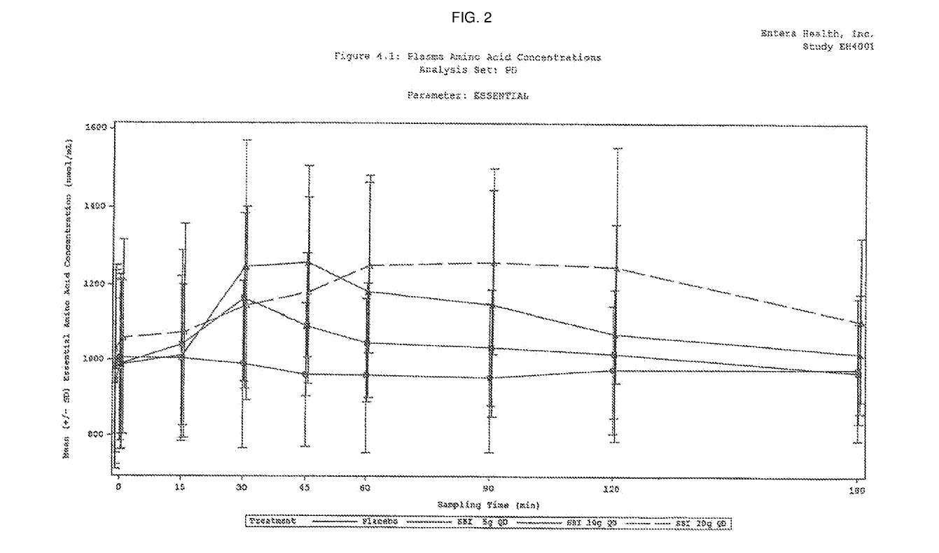

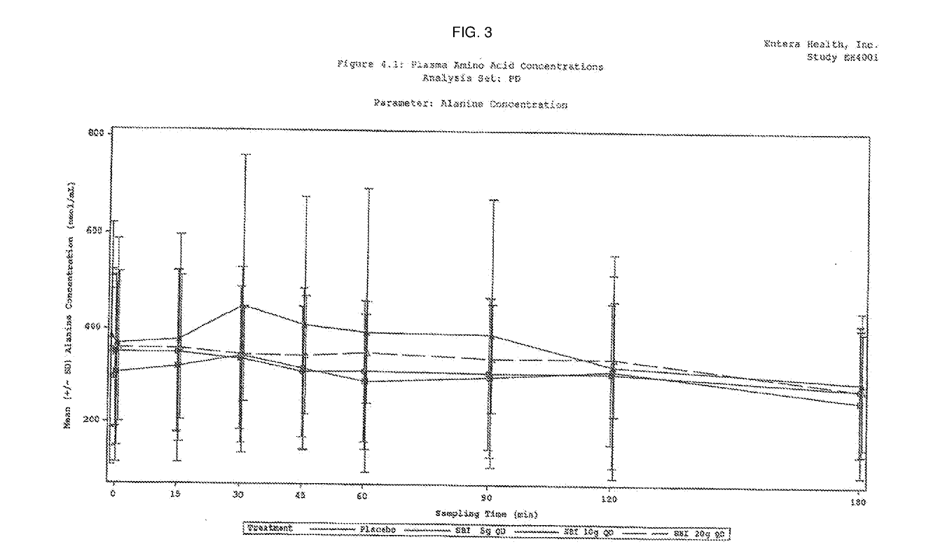

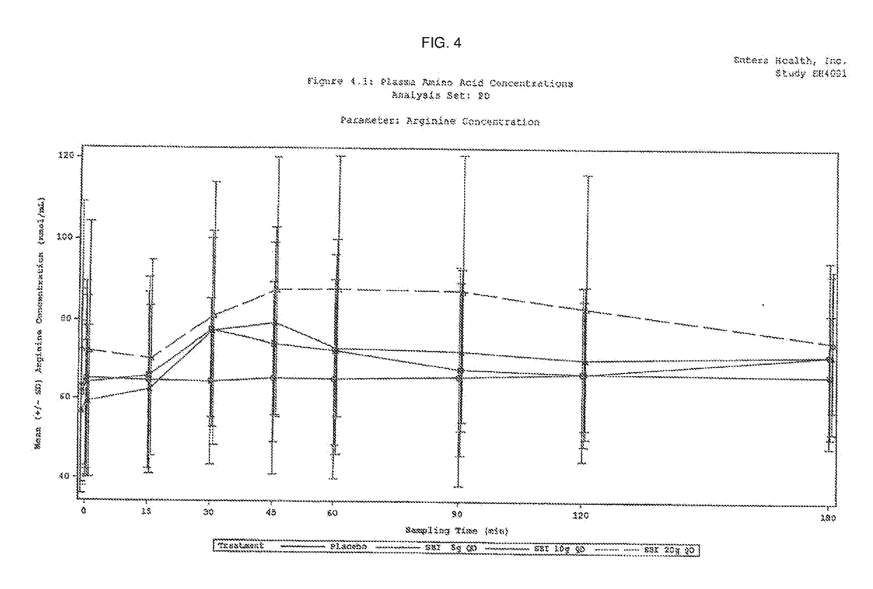

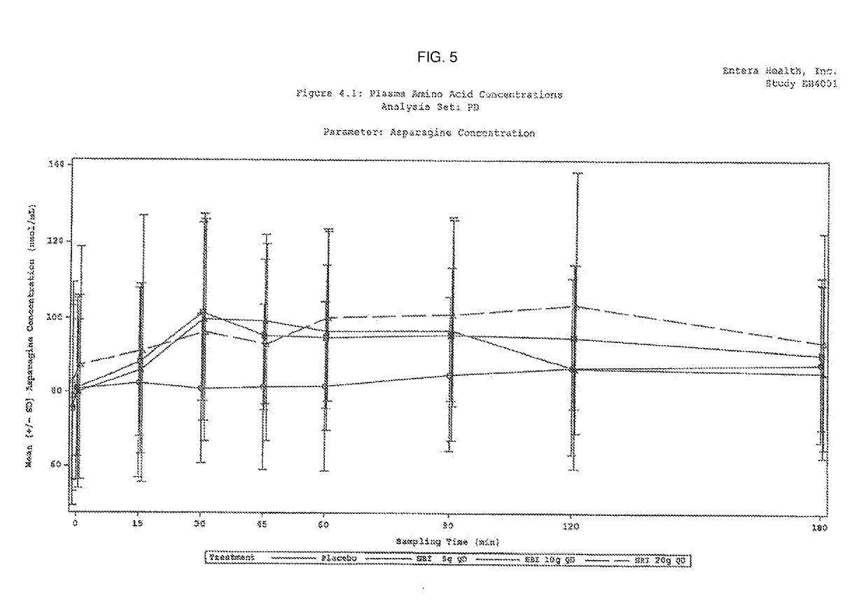

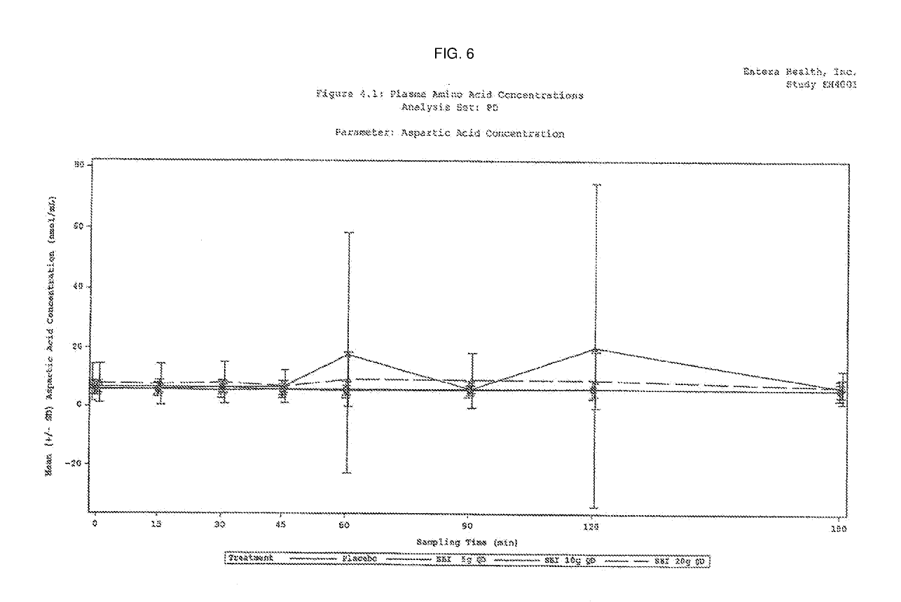

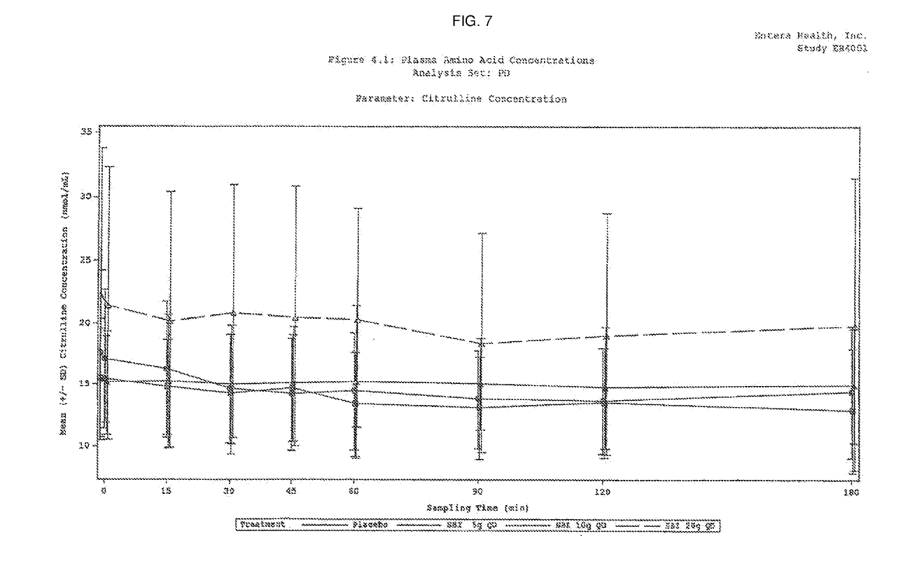

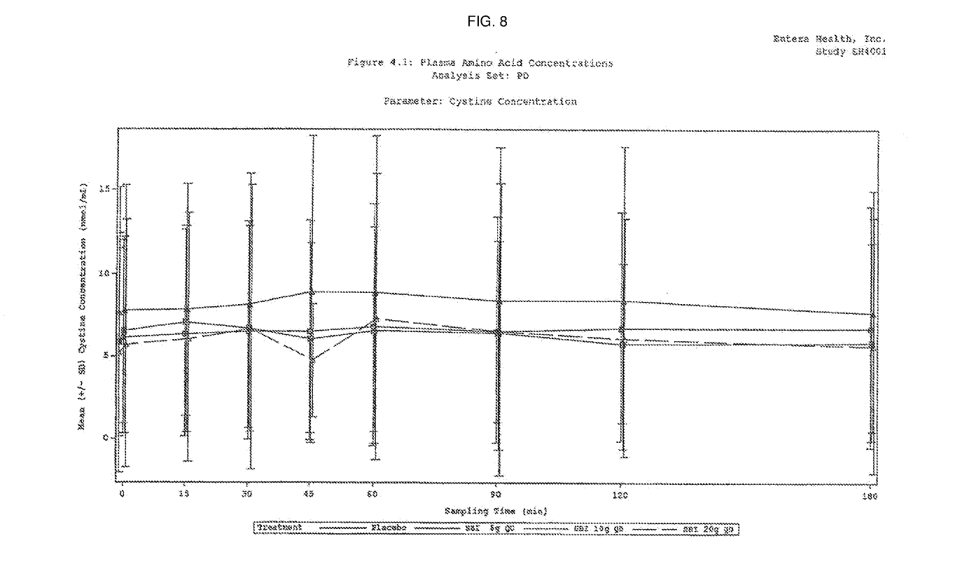

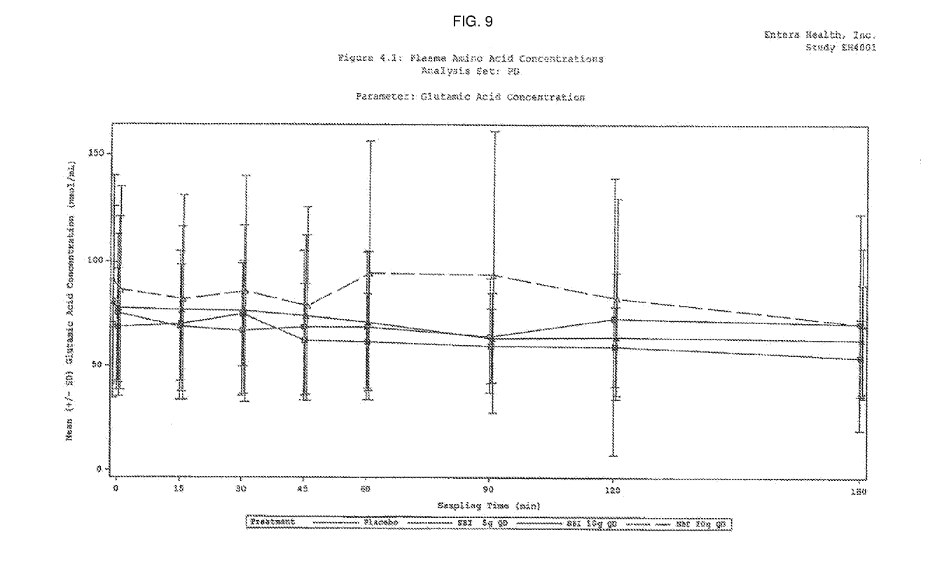

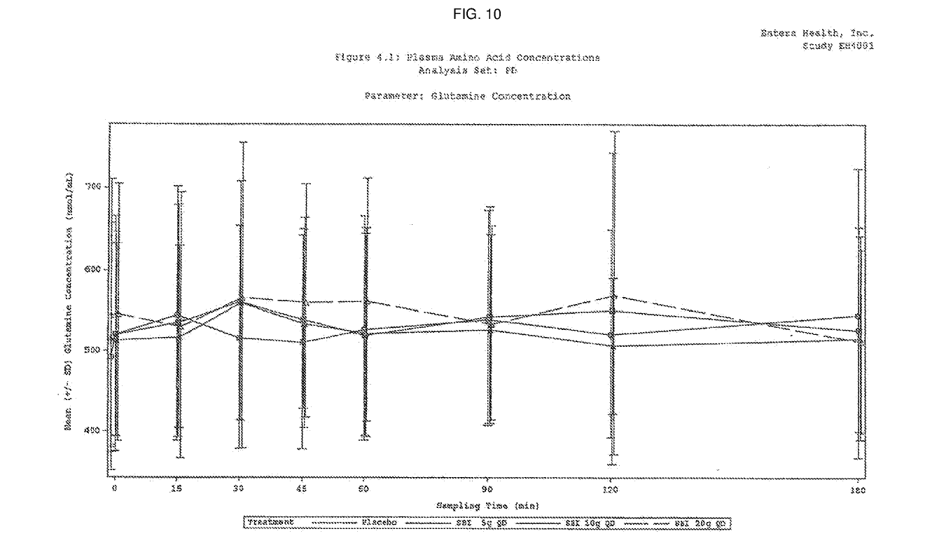

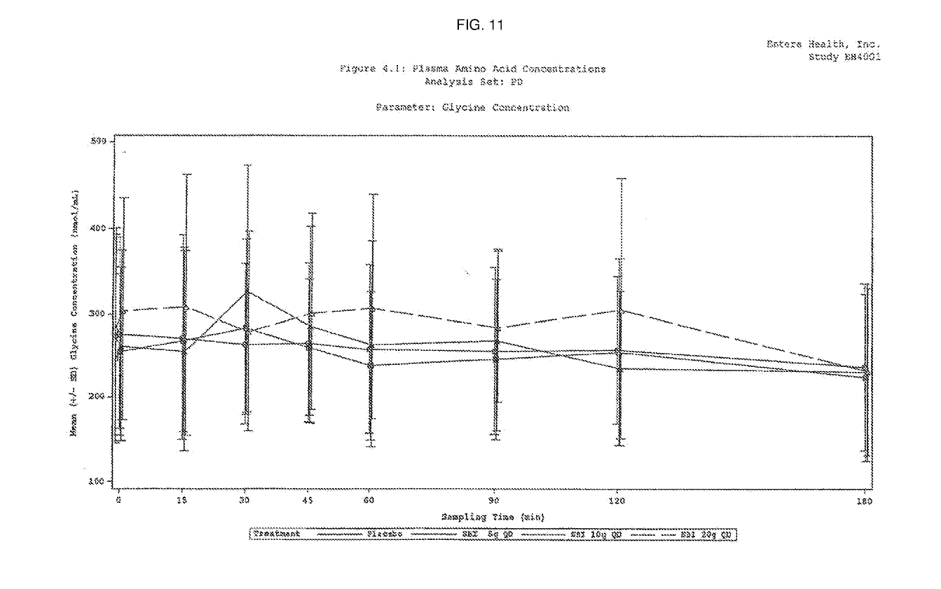

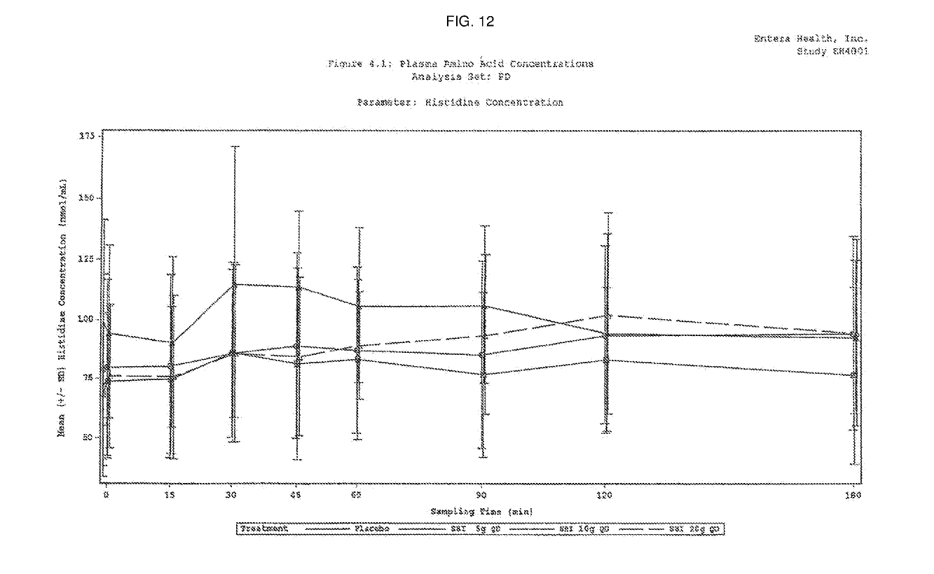

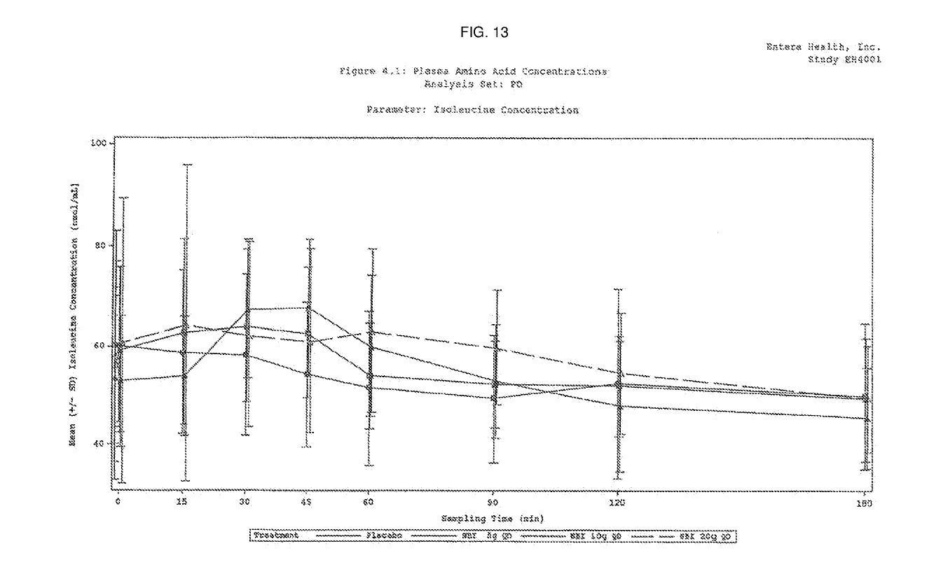

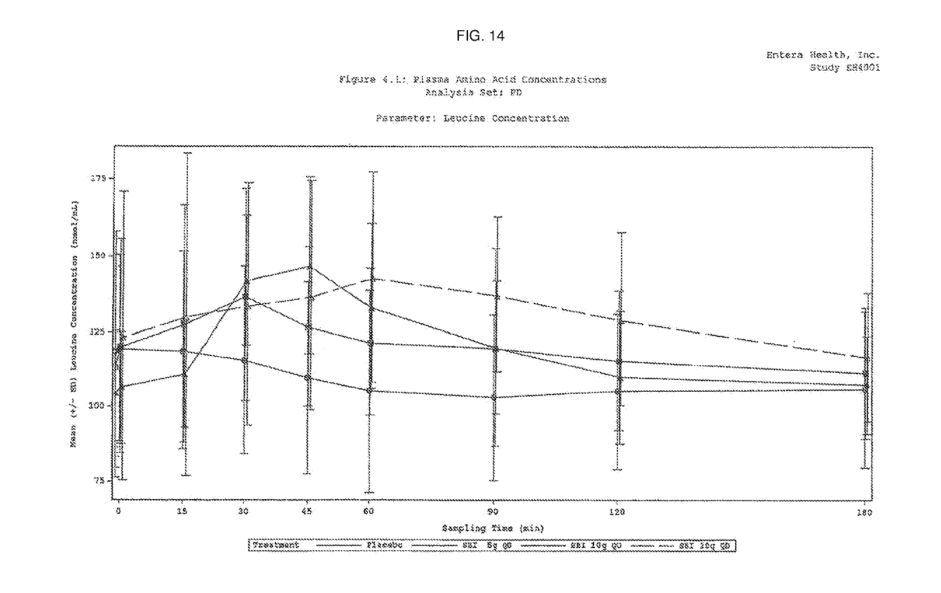

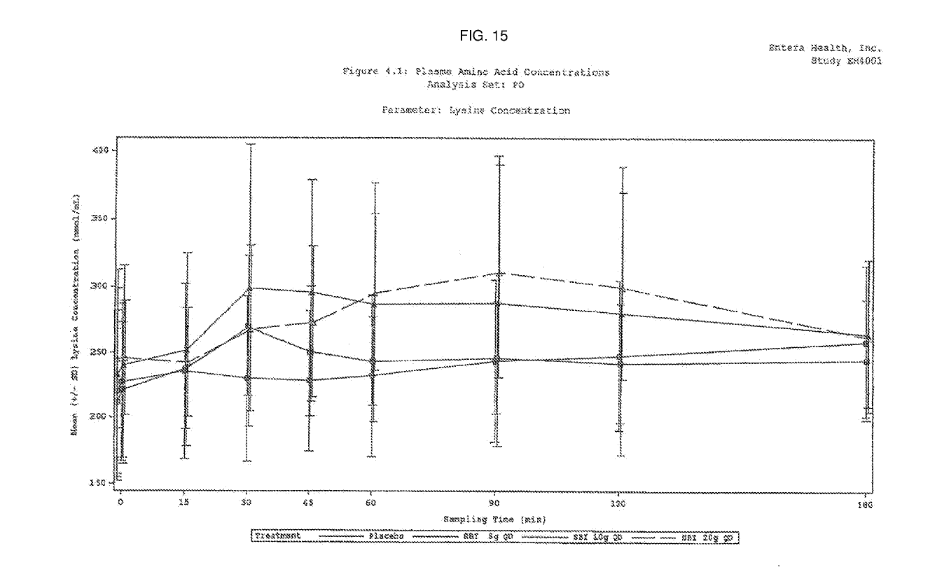

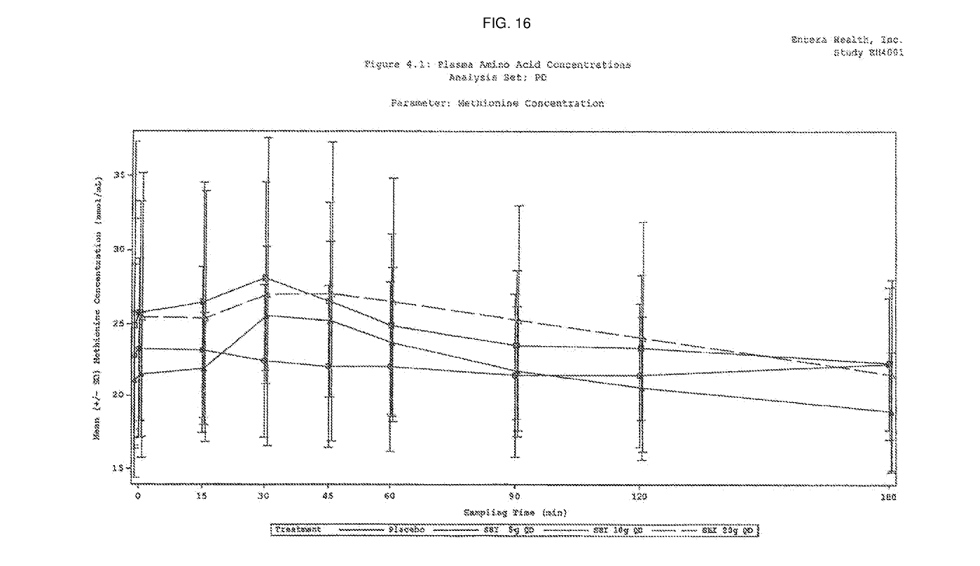

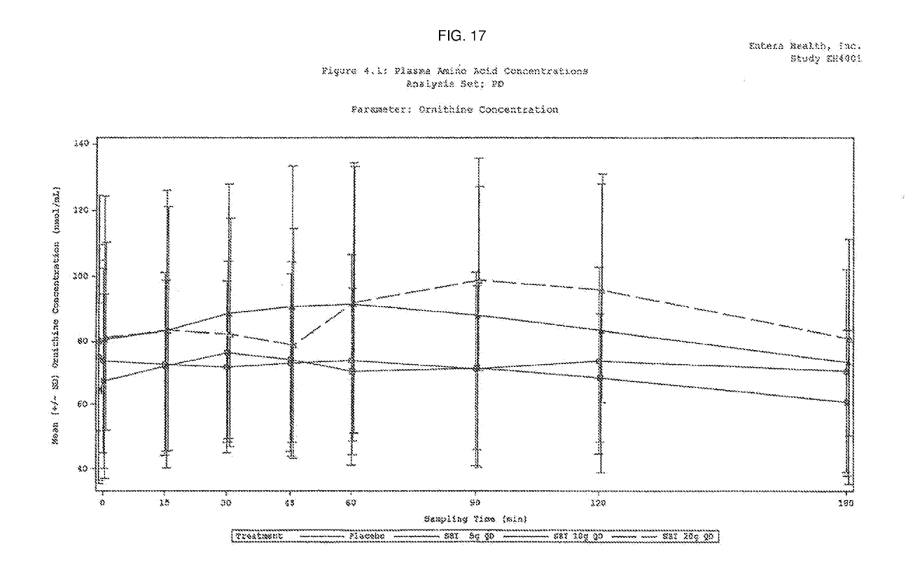

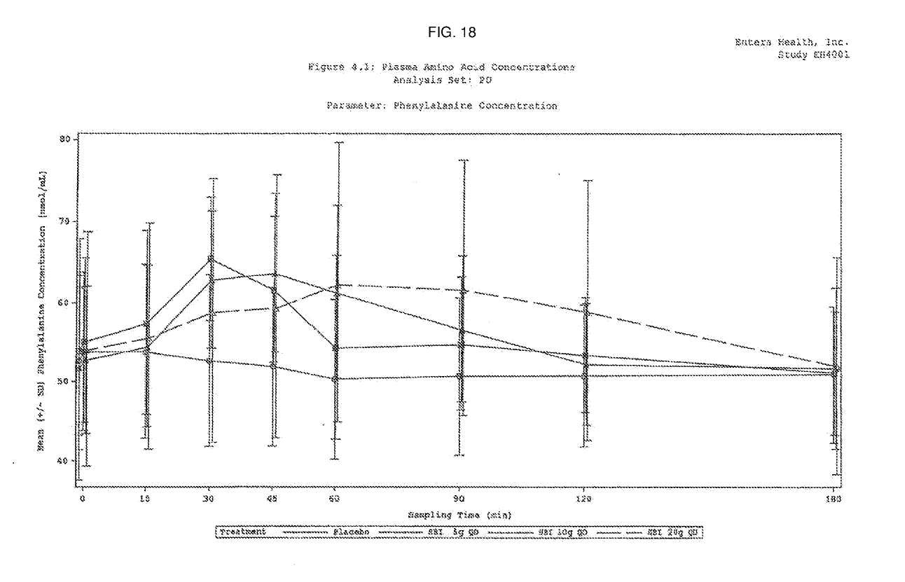

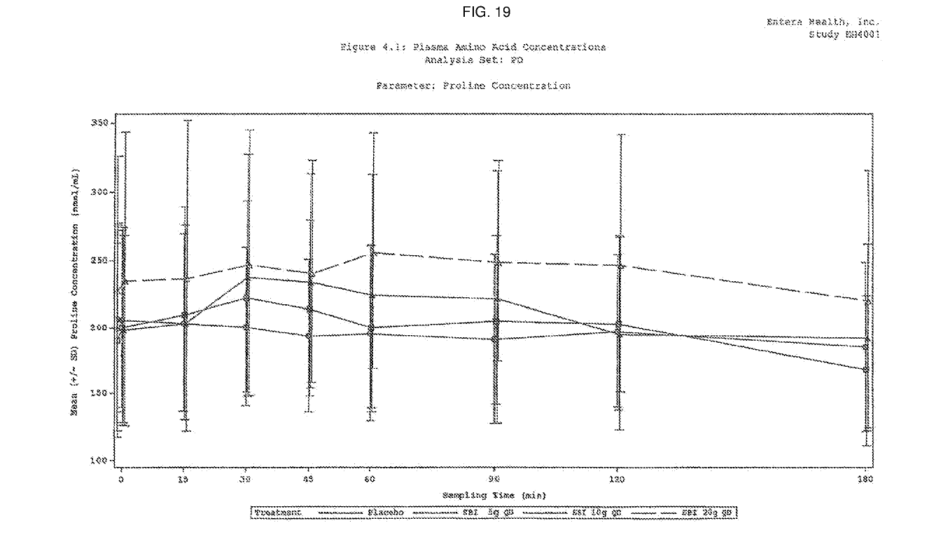

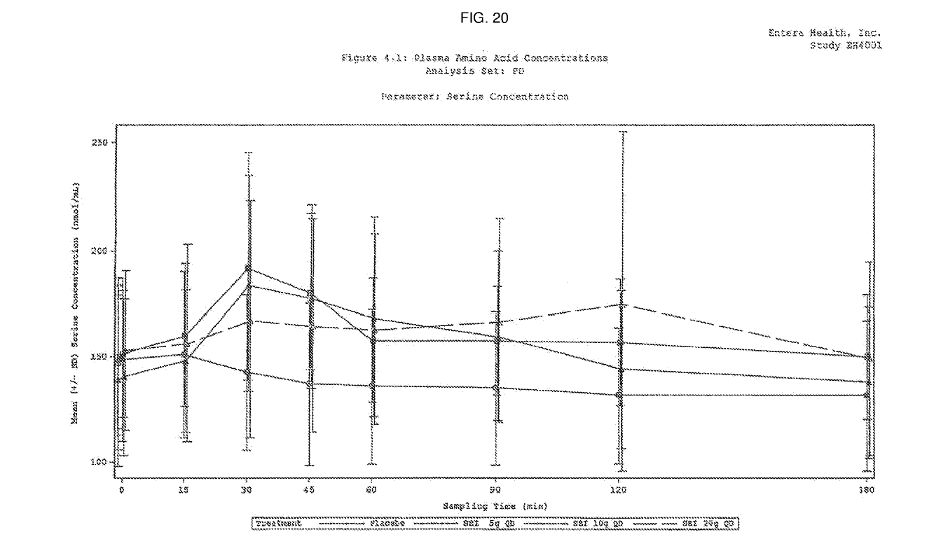

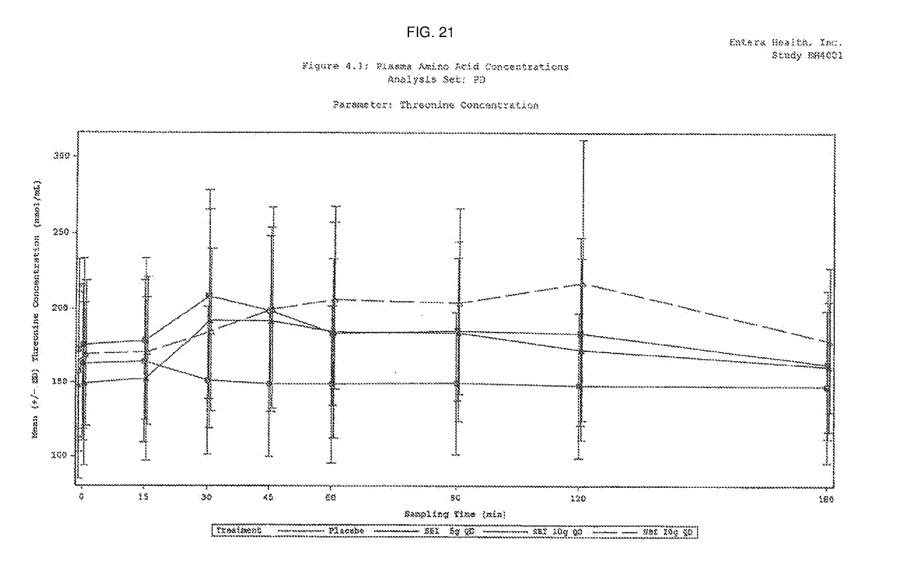

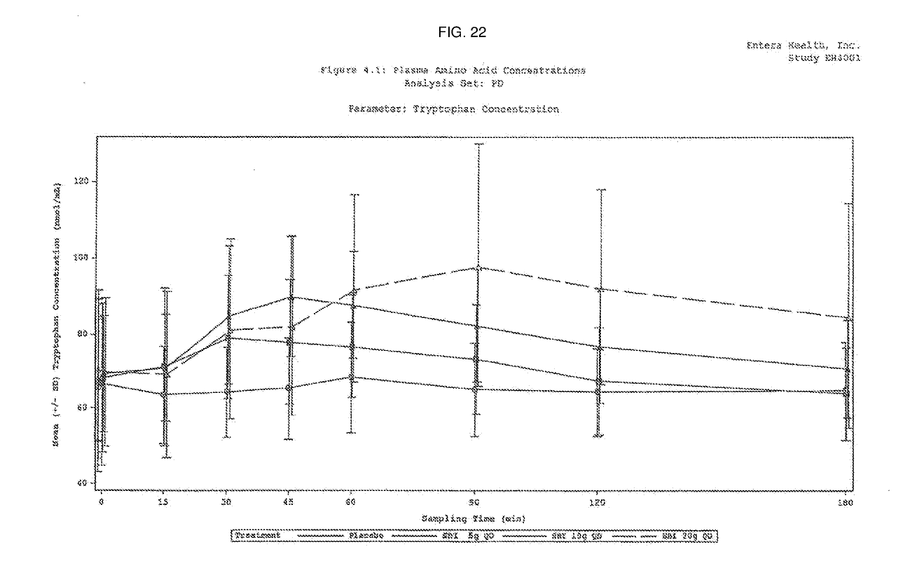

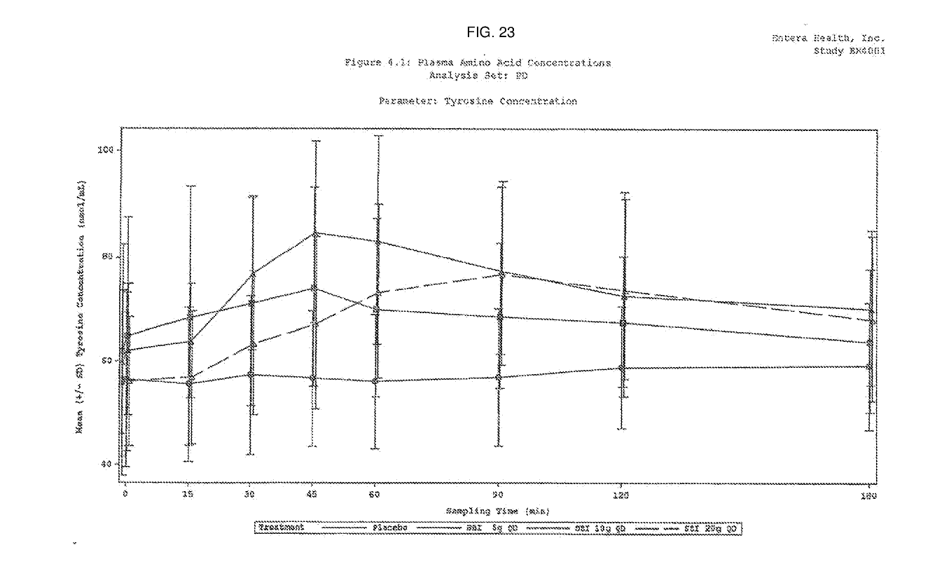

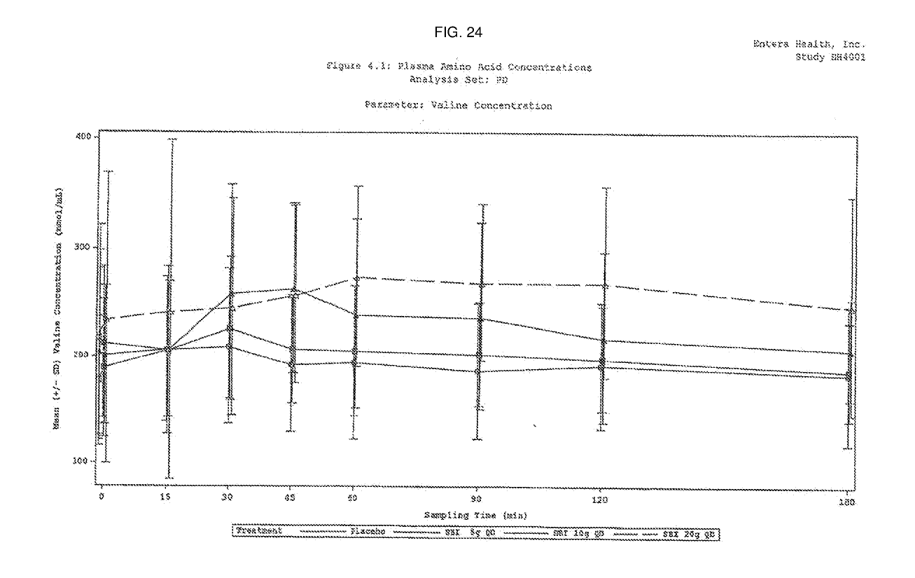

### DETAILED DESCRIPTION OF THE INVENTION

According to the invention. Applicant has provided herein a pharmaceutical composition comprising immunoglobulin components purified and concentrated from animal plasma which provide a change of digestability profile, resulting in sustained plasma concentrations of essential amino acids and, including tryptophan and leucine. The compositions are useful for retaining muscle mass patients with involuntary weight loss, as well as for building body mass in athletes. While bovine serum immuglobulin is preferred for use in the invention, the invention contemplates the use of immunoglobulin from any source.

According to the invention, the IgG compositions for use in the invention include gamma-globulin isolated from animal sources such as serum, plasma, egg, or milk. As used herein with reference to the composition of the invention, the terms “plasma”, “globulin”, “gamma-globulin”, and “immunoglobulin” will all be used. These are all intended to describe a composition purified from animal sources including blood, egg, or milk-which retains the Fc region of the immunoglobulin molecule. This also includes transgenic recombinant immunoglobulins purified from transgenic bacteria, plants or animals. This can be administered by spray-dried plasma, or globulin which has been further purified therefrom, or any other source of serum globulin which is available.

The IgG composition of this invention preferably consists of a plasma fraction where the majority of fibrin and albumin have been removed from the plasma. One source of immunoglobulin is EnteraGam® available from Entera Health Inc. EnteraGam is derived from purified bovine serum (SBI) and typically consists of 50% or more by weight IgG and 10% or less by weight albumin. Suitable IgG compositions of the invention include plasma fractions consisting of 50%, 55%, 60%, 65%, 70%, 75%, 80%, 85%, 90%, or 95% by weight IgG. Other suitable IgG compositions of the invention include plasma fractions consisting of 1%, 2%, 3%, 4%, 5%, 6%, 7%, 8%, 9%, or 10% by weight albumin.

Globulin may be purified according to any of a number of methods available in the art, including those described in Akita, B. M. and S, Nakai. 1993. Comparison of four purification methods for the production of immunoglobulins from eggs laid by hens immunized with an enterotoxigenic E. coli strain. Journal of Immunological Methods 160:207-214; Steinbuch, M. and R. Audran. 1969, The isolation of IgG from mammalian sera with the aid of caprylic acid. Archives of Biochemistry and Biophysics 134:279-284; Lee, Y., T. Aishima, S. Nakai, and J. S. Sim, 1987. Optimization for selective fractionation of bovine blood plasma proteins using polyethylene glycol. Journal of Agricultural and Food Chemistry 35:958-962; Poison, A., G. M. Potgieter, J. F. Langier, G. E. F. Mears, and F. J. Toubert. 1964. Biochem. Biophys. Acta. 82:463-475.

Animal plasma from which immunoglobulin or other plasma fractions may be isolated include porcine, bovine, ovine, poultry, equine, or goat plasma. Additionally, applicants have identified that cross species sources of the gamma globulins still provides the effects of the invention.

Concentrates of the product can be obtained by spray drying, lyophilization, or any other conventional drying method so long as it does not inactivate or damage the proteins. The concentrates may also be used in their liquid or frozen form.

The active ingredient may also be microencapsulated, protecting and stabilizing from high temperature, oxidants, enteric digestion, etc. The pharmaceutical compositions of the invention can be in tablets, capsules, ampules for oral use, granulate powder, cream, both as a unique ingredient and associated with other excipients or active compounds, or even as a feed additive.

One method of achieving a gamma-globulin composition concentrate of the invention is as follows although the globulin may be delivered as a component of plasma.

The immunoglobulin concentrate is derived from animal blood. The source of the blood can be from any animal that has blood which includes plasma and immunoglobulins. For convenience, blood from beef, pork, and poultry processing plants is preferred. Anticoagulant is added to whole blood and then the blood is centrifuged to separate the plasma. Any anticoagulant may be used for this purpose, including sodium citrate and heparin. Persons skilled in the art can readily appreciate such anticoagulants. Calcium or other suitable reagent to react with fibrinogen is then added to the plasma to promote precipitation, or to facilitate the removal of fibrinogen; however other methods are acceptable. This mixture is then centrifuged to remove the fibrin portion.

Once the fibrin is removed from plasma resulting in serum, the serum can be used as a principal source of Ig. The fibrinogen depleted plasma is next treated with an amount of salt compound or polymer sufficient to precipitate the albumin or globulin fraction of the plasma. Examples of phosphate compounds which may be used for this purpose include all polyphosphates, including sodium hexametaphosphate and potassium polyphosphate. The globulin may also be isolated through the addition of polyethylene glycol or ammonium sulfate.

Following the addition of the phosphate compound, the pH of the plasma solution is lowered to stabilize the albumin precipitate. The pH should not be lowered below 3.5, as this will cause the proteins in the plasma to become damaged. Any type of acid can be used for this purpose, so long as it is compatible with the plasma solution. Persons skilled in the art can readily ascertain such acids. Examples of suitable acids include, but are not limited to, acetic acid, H₂SO₄, citric acid, and H₂PO₄. The acid is added in an amount sufficient to lower the pH of the plasma to the designated range. Generally, this amount will range from a ratio of about 1:4 to 1:2 acid to plasma. The plasma is then centrifuged to separate the globulin fraction from the albumin fraction.

The next step in the process is to raise the pH of the globulin fraction with a base until it is no longer corrosive to separation equipment. Acceptable bases for this purpose include NaOH, KOH, and other alkaline bases. Such bases are readily ascertainable by those skilled in the art. The pH of the globulin fraction is raised until it is within a non-corrosive range which will generally be between 5.0 and 9.0.

The final immunoglobulin concentrate can optionally be spray-dried or lyophilized into a powder. The powder allows for easier packaging and the product remains stable for a longer period of time than the raw globulin concentrate in liquid or frozen form. The immunoglobulin concentrate powder has been found to contain approximately 40-55% by weight IgG, 1-2% by weight IgA, and 6-8% by weight IgM. At a minimum, the immunoglobulin concentrate of the invention should contain at least 40% by weight IgG, with at least 50% by weight IgG being preferred, and at least 55% by weight being most preferred. In some embodiments, the immunoglobulin concentrate of the invention contains 40%, 41%, 42%, 43%, 44%, 45%, 46%, 47%, 48%, 49%, 50%, 51%, 52%, 53%, 54%, or 55% by weight IgG. In some embodiments, the immunoglobulin concentrate of the invention contains 1%, 1.5%, or 2% by weight IgA. In some embodiments, the immunoglobulin concentrate of the invention contains 6%, 6.5%, 7%, 7.5%, or 8% by weight IgM.

In addition to administration with conventional carriers, active ingredients may be administered by a variety of specialized delivery drug techniques which are known to those of skill in the art.

Those skilled in the medical arts will readily appreciate that the doses and schedules of the immunoglobulin and protein intake will vary depending on the age, health, sex, size and weight of the patient rather than administration, etc. These parameters can be determined for each system by well-established procedures and analysis e.g., in phase I, II, and III clinical trials.

In accordance with certain embodiments of the invention, the immunoglobulin concentrate (IgC) is administered orally or by tube feeding at a dose of at least 20 g to provide at least 10 g IgG. In another embodiment, the IgC is administered in a dose of at least 30 g to provide at least 15 g IgG. In another embodiment, the IgC is administered in a dose of at least 40 g to provide at least 20 g IgG. In yet another embodiment, the IgC is administered in a dose of at least 50 g to provide at least 25 g IgG. Other doses of IgC of the invention include 25 g, 35 g, 45 g, and 55 g. The doses may be provided once daily, or divided and administered twelve hours apart, for example, to provide consistent amino acid serum levels throughout the day.

The globulin concentrate can be combined with a pharmaceutically acceptable carrier such as a suitable liquid vehicle or excipient and an optional auxiliary additive or additives. The liquid vehicles and excipients are conventional and are commercially available. Illustrative thereof are distilled water, physiological saline, aqueous solutions of dextrose and the like.

The immunoglobulin composition of this invention may be administered alone or in conjunction with other therapeutic agents, either in a kit for combination therapy or combined in the same pharmaceutical dosage form. Such additional therapeutic agents would include, but are not limited to, antibiotics, analgesics, antivirals, NSAIDS, corticosteroids, etc. Any type of medication may be used in this regard so long as it is compatible with the plasma fraction and does not inactivate or damage the proteins in the IgC.

In general, in addition to the active compounds, the pharmaceutical compositions of this invention may contain suitable excipients and auxiliaries which facilitate processing of the active compounds into preparations which can be used pharmaceutically. Oral dosage forms include tablets, caplets, and capsules.

The pharmaceutical preparations of the present invention are manufactured in a manner which is itself well known in the art. For example the pharmaceutical preparations may be made by means of conventional mixing, granulating, dissolving, and lyophilizing processes. The processes to be used will depend ultimately on the physical properties of the active ingredient used.

Suitable excipients are, in particular, fillers such as sugars for example, lactose or sucrose, mannitol or sorbitol, cellulose preparations and/or calcium phosphates, for example, tricalcium phosphate or calcium hydrogen phosphate, as well as binders such as starch, paste, using, for example, maize starch, wheat starch, rice starch, potato starch, gelatin, gum tragacanth, methyl cellulose, hydroxypropylmethylcellulose, sodium carboxymethylcellulose, and/or polyvinyl pyrrolidone. If desired, disintegrating agents may be added, such as the above-mentioned starches as well as carboxymethyl starch, cross-linked polyvinyl pyrrolidone, agar, or alginic acid or a salt thereof, such as sodium alginate. Auxiliaries are flow-regulating agents and lubricants, for example, such as silica, talc, stearic acid or salts thereof, such as magnesium stearate or calcium stearate and/or polyethylene glycol. For this purpose concentrated sugar solutions may be used, which may optionally contain gum arabic, talc, polyvinylpyrrolidone, polyethylene glycol and/or titanium dioxide, lacquer solutions and suitable organic solvents or solvent mixtures. In order to produce coatings resistant to gastric juices, solutions of suitable cellulose preparations such as acetyl cellulose phthalate or hydroxypropylmethylcellulose phthalate, dyestuffs and pigments may be added to the tablet, for example, for identification or in order to characterize different combination of compound doses.

Other pharmaceutical preparations which can be used orally include capsules made of gelatin, as well as soft, sealed capsules made of gelatin and a plasticizer such as glycerol or sorbitol. The capsules can contain the active compounds in the form of granules which may be mixed with fillers such as lactose, binders such as starches, and/or lubricants such as talc or magnesium stearate and, optionally, stabilizers. In soft capsules, the active compounds are preferably dissolved or suspended in suitable liquids; such as fatty oils, liquid paraffin, or liquid polyethylene glycols. In addition stabilizers may be added.

The therapeutic administration of oral doses of globulin concentrate at doses of at least 20 g were found to maintain peak amino acid levels for at least 15 minutes, and up to 180 minutes following administration. Without limiting themselves to any particular theory, the present inventors believe the sustained amino acid levels may be due to the presence of proteins in the composition that are inhibiting digestion of the amino acids, i.e. anti-proteases.

The IgC of the invention may be administered to any animal that requires additional protein. In one embodiment of the invention, the IgC is administered to a malnourished or sick animal, i.e. an animal that is receiving <75% of its daily protein requirement. In one embodiment of this invention, the animal receives <25% of its daily protein requirement during the treatment period. In another embodiment of the invention the IgC is administered to an athlete or bodybuilder to increase systemic protein content.

The following example is intended to illustrate certain embodiments of the invention without limitation.

### EXAMPLE

### Effects of Bovine Immunoglobulin Concentrate on Plasma Amino Acid Concentrations

Experimental Design and Method:

Results

While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

Having described the invention with reference to particular compositions, theories of effectiveness, and the like, it will be apparent to those of skill in the art that it is not intended that the invention be limited by such illustrative embodiments or mechanisms, and that modifications can be made without departing from the scope or spirit of the invention, as defined by the appended claims. It is intended that all such obvious modifications and variations be included within the scope of the present invention as defined in the appended claims. The claims are meant to cover the claimed components and steps in any sequence which is effective to meet the objectives there intended, unless the context specifically indicates to the contrary.

What is claimed is:

1. A method of increasing plasma amino acid concentrations in an animal comprising: orally administering a dose of at least 20 g of immunoglobulin concentrate (IgC) to an animal.

2. The method of claim 1 wherein the IgC includes at least 50% by weight IgG.

3. The method of claim 1 wherein the IgC is administered to an animal that is malnourished.

4. The method of claim 1 wherein the IgC is administered to an athlete or bodybuilder.

5. The method of claim 1 wherein the IgC is serum bovine immunoglobulin (SBI).

6. The method of claim 5 wherein the IgC contains 10% or less albumin.

7. The method of claim 1 wherein following administration the animal experiences peak amino acid plasma concentrations for at least 15 minutes.

8. The method of claim 7 wherein following administration the animal experiences peak amino acid plasma concentrations for about 120 minutes.

9. The method of claim 7 wherein the animal experiences peak tryptophan and leucine plasma concentrations for at least 15 minutes.

10. The method of claim 1 wherein the animal is administered a dose of IgC of at least 30 g.

11. The method of claim 1 wherein the animal is administered a dose of IgC of at least 40 g.

12. The method of claim 1 wherein the animal is administered a dose of IgC of at least 50 g.
